(19) United States
(12) Patent Application Publication
(10) Pub. No.: US 2020/0138996 A1
(43) Pub. Date: May 7, 2020

(54) APPARATUS AND METHOD FOR GENERATING PROGRAMMABLE FRAGRANCE

(71) Applicants: CHEYANG CHEN, Claremont, CA (US); YU CHANG, Taipei (TW)

(72) Inventors: CHEYANG CHEN, Claremont, CA (US); YU CHANG, Taipei (TW)

(21) Appl. No.: 16/676,421

(22) Filed: Nov. 6, 2019

Related U.S. Application Data
(60) Provisional application No. 62/756,079, filed on Nov. 6, 2018.

(51) Int. Cl. A61L 9/03

(52) U.S. Cl. CPC A61L 9/03; A61L 2209/13; A61L 2209/11

(57) ABSTRACT

In one aspect, a programmable fragrance generating apparatus may include a control center, a plurality of liquid fragrance receiving holes, and a receiving space for at least one EMF platform, wherein the EMF platform is electrically connected with the control center and has a plurality of reservoirs to receive different liquid fragrances from the fragrance receiving holes; each liquid fragrance is controlled by the control center to transport from each reservoir to one or more driving electrodes on the EMF cartridge to mix with one or more other liquid fragrances, which can be further transported to one or more microheater electrodes, atomizers, or nebulizers for fragrance releasing by evaporation at a predetermined temperature.

### CROSS-REFERENCE TO RELATED APPLICATIONS

This application claims priority under 35 U.S.C. § 119(e) to U.S. Provisional Patent Application Ser. No. 62/756,079, filed on Nov. 6, 2018, the entire contents of which are hereby incorporated by reference.

### FIELD OF THE INVENTION

The present invention relates to an apparatus and method for generating fragrance, and more particularly to an electro-microfluidic (EMF) platform configured to manipulate droplets with different fragrances to generate programmable and reproduceable fragrances according to the user's preferences.

### BACKGROUND OF THE INVENTION

Olfactory sense is one of the five senses of human beings. We have long developed numerous ways to communicate and transfer messages mainly by our senses of sight and hearing through images, literatures, music and sounds because of their ease of documentation and reproduction. Although the odor of the environment significantly influences our mood and possibly delivers messages by recalling memory or triggering emotions, the smell-based communication requires an effective way to encode, record and reproduce the odor and fragrance. Unlike images that are the combinations of three primary colors and music that is composed of seven basic notes, the odor has much more complicated chemical compositions which are hard to thoroughly decomposed and regenerated.

Therefore, there remains a need for a new and improved apparatus to generate programmable and reproduceable fragrances to overcome the problems stated above.

### BRIEF DESCRIPTION OF THE DRAWINGS

FIGS. 1a and 1b are schematic views of driving dielectric essential oil droplets by dielectrophoresis between parallel plates of the EMF platform. FIG. 1a is a cross section of the EMF platform showing the top plate containing an unpatterned electrode and a hydrophobic coating, and the bottom plate holding patterned electrodes, dielectric and hydrophobic layers; FIG. 1b is a top view of the square driving electrodes on the bottom plate.

FIGS. 2a to 2c show an example of the electrode design on the bottom plate of the EMF platform. FIG. 2a is the bottom plate having mainly the reservoir electrodes, driving electrodes and microheater electrodes; FIG. 2b is the center 3×4 driving electrode array for essential oil droplets mixing; and FIG. 2c is the design of the microheater electrodes, for example, for fragrance releasing by evaporation at controllable temperature and power.

FIGS. 3a to 3f illustrate essential oil droplet manipulations. FIG. 3a shows generating an orange essential oil droplet (40 nL) from the left reservoir (3 μL); FIG. 3b shows transporting the orange essential oil droplet above the clove essential oil droplet generated from the lower reservoir; FIGS. 3c and 3d illustrate mixing the two essential oil droplets by driving the merged droplet along a loop path; FIG. 3e shows splitting the mixture droplet; and FIG. 3f shows preparing droplets with different orange to clove essential oil ratios 1:0, 1:2, 0:1 and 1:1.

FIG. 4 is an example showing that the droplet velocity is adjustable of orange and clove essential oils at different voltages.

FIGS. 5a to 5d show fragrance releasing by evaporating the essential oil on the microheater electrodes. FIG. 5a shows sequentially generating eight orange essential oil droplets (each volume 40 nL) from the left reservoir; FIG. 5b shows merging the droplets on the microheaters; and FIGS. 5c and 5d show fragrance releasing when the microheaters 2 and 4 in FIG. 2c are energized by a DC voltage.

FIG. 6 illustrates the fragrance release rate tunable by adjusting the voltage.

FIG. 7 is a schematic view of a programmable fragrance generating apparatus in the present invention.

FIG. 7a is a block diagram of the control center of the programmable fragrance generating apparatus in the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

While decoding and documenting the odor is still challenging, the present invention is configured to encode and reproduce the fragrance based on commercially available top, middle and base essential oils. Arbitrary fragrance is obtainable on an electro-microfluidic (EMF) platform by generating and mixing individual essential oil droplets with the volume of 40 nL by dielectrophoresis, for example. In addition, the mixture of the essential oil droplets at a specific encoded ratio is subsequently released by resistive heating on the EMF platform at a tunable evaporation rate. For tiny droplet volumes, the released fragrance can be rapidly changed along with time, and the EMF platform in the present invention is capable of encoding and releasing fragrance at a programmable sequence and rate that actively alters the environmental fragrance for either entertainment or medical (e.g., aromatherapy) purposes.

The liquid mixtures can also be released in other forms including liquid and gas phases. For example, the mixture droplets can be released by nebulizing or atomization. With integrated atomizers and nebulizers, the EMF platform in the present invention would become an ideal platform for programmable preparations of the mixture of chemicals and drugs for other breathing treatments.

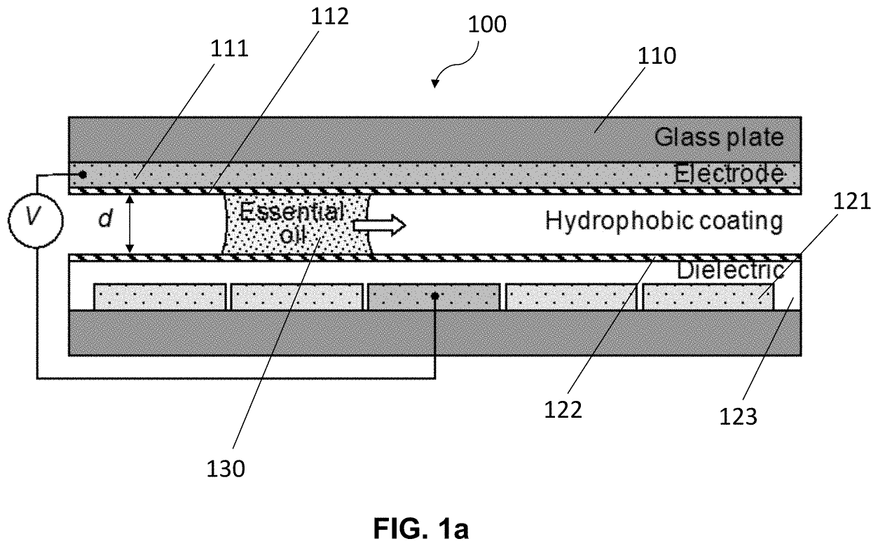

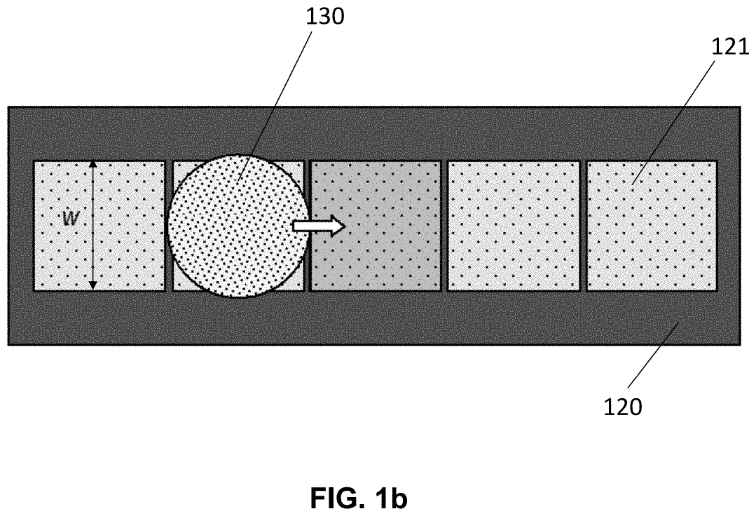

In one aspect, as shown in FIGS. 1a and 1b, an electro-microfluidic (EMF) platform 100 used in the present invention may include a top plate 110 and a bottom plate 120, which are parallel with each other. A plurality of bottom electrodes 121 are patterned on the bottom plate 120 while a top electrode 111 disposed on the top plate 110 may be unpatterned and covered by a top hydrophobic coating 112. The bottom electrodes 121 are covered by a bottom hydrophobic coating 122 and a dielectric layer 123. With the parallel-plate configurations, the electro-microfluidic (EMF) platform 100 is configured to offer electric forces for a variety of manipulations, such as (1) multiphase oil droplets and gas/plasma bubbles driving, and (2) cross-scale objects of droplets (mm) and suspended particle/cell (μm) actuation. In one embodiment, the top/bottom plate can be made by glass.

Dielectric droplets are in general driven by dielectrophoresis that draws bulk dielectric liquids of higher relative permittivity (liquid) into a strong electric field region of lower relative permittivity (air). As illustrated in FIGS. 1a and 1b, when a voltage V is applied between w-wide and d-spaced parallel top and bottom electrodes, a dielectric droplet would be attracted toward the strong electric field region by the dielectrophoresis force:

$${F}_{OFF} = \frac{{ɛ}_{0} ( {ɛ}_{L} - 1 ) W}{2 d} {V}^{2} ,$$

where ε₀ (8.85×10⁻¹² F/m) is the permittivity of vacuum, and ε_{L} is the relative permittivity of the liquid. In one embodiment, the dielectrophoresis force is used to manipulate a dielectric essential oil droplet 130 for programmable fragrance production in the present invention.

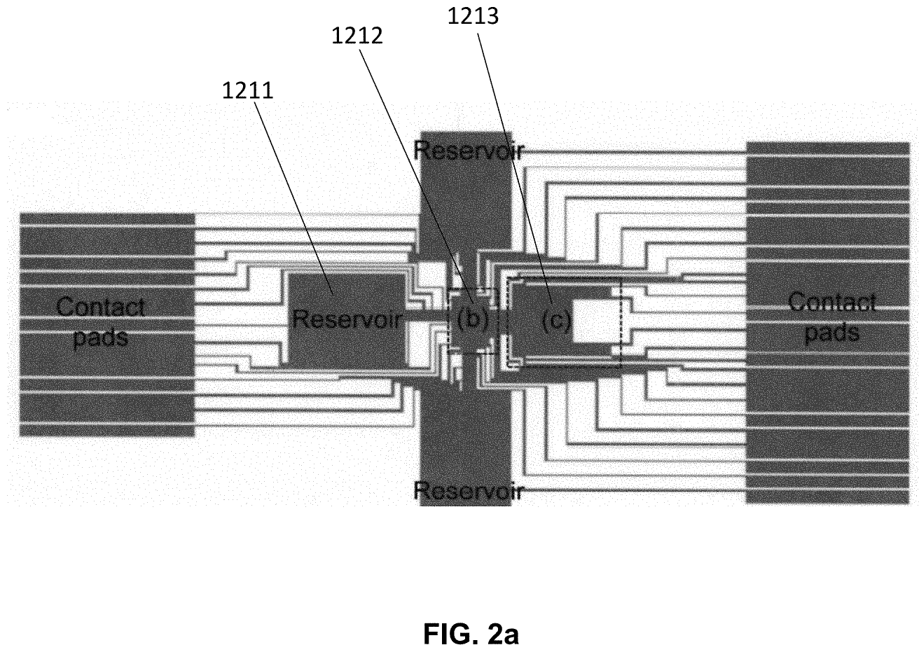

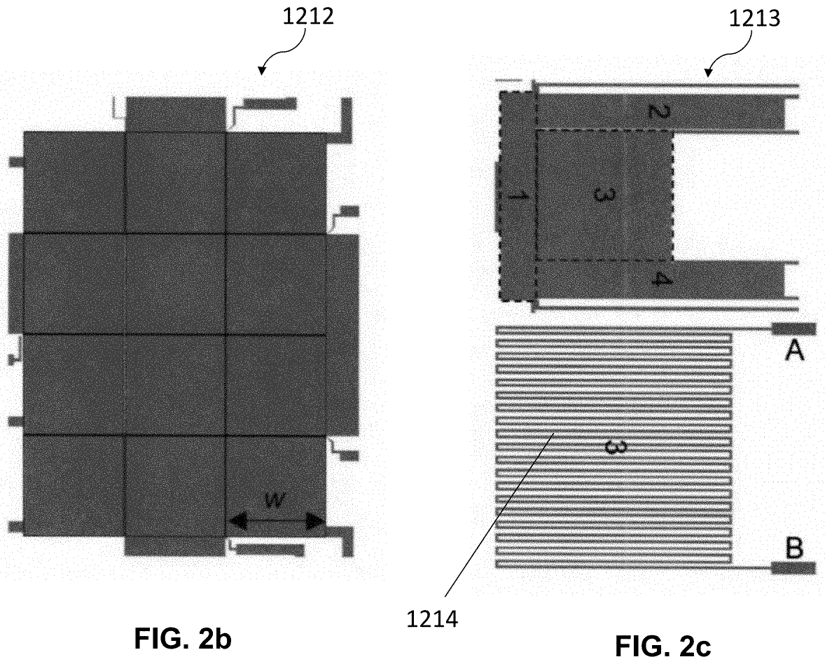

In one embodiment, the design of the electrodes 121 on the bottom plate 120 of the EMF platform 100 is shown in FIG. 2a. The upper and lower portions of the plate may include the contact pads that connected to the outer electronic circuits and the electrodes at the center region of the plate, where three types of electrodes are designed, including one or more reservoir electrodes 1211, driving electrodes 1212 and microheater electrodes 1213. The reservoir electrodes 1211 are patterned on the bottom plate 120 to accommodate different elementary essential oils for example. Droplets of the essential oils are generated from their own reservoirs and transported to the center area with an array of the driving electrodes 1212 as shown in FIG. 2b for droplets mixing to produce adjustable and encoded fragrances. The mixture of the essential oil droplets is subsequently transported to the microheater electrodes 1213 as shown in FIG. 2c.

In an exemplary embodiment, each microheater 1213 may include a meandered electrode line pattern 1214 to provide a programmable essential oil release rate by tuning the input Joule heating power and the resulting temperature. The microheater electrodes 1213 have two electric terminals (A, B) and when applying an equal electric potential on the two terminals, the electric field between the top and bottom plates generates dielectrophoresis force and drives the dielectric essential oil droplets 130 between plates 110 and 120. When an electric potential is applied between the two terminals, the electrode behaves as a microheater and generates heats based on the Joule's law. Other releasing mechanisms, including atomizers and nebulizers, can be integrated on the EMF platform 100 as well.

To fabricate the EMF platform 100, the parallel-plate EMF platform is designed and fabricated as shown in FIGS. 1a-1b and 2a-2c. For example, the reservoir electrodes 1211, driving electrodes 1212 and microheater electrodes 1213 are patterned on the bottom plates 120 by photolithography and wet (or dry) etching of a conductive thin film (e.g., ITO (indium tin oxide, thickness 260 nm), conductive oxide, metal, conductive polymer) on glass. Depending on the targeted droplet size, in one embodiment, the width (w in FIG. 1b) of the square driving electrodes can be 1 mm, and the width of the meandered lines 1214 of the microheaters can be 20 μm. In another embodiment, the lengths in the heating area can be from 100 to 1000 mm for proper microheater electrodes 1213, and the resulting entire resistances are about several to hundreds of kΩ. The top/bottom hydrophobic layers (112, 122), e.g., fluoropolymer, Cytop, Teflon, can be spun or sprayed to make the surface hydrophobic. In one embodiment, the thickness of the hydrophobic layer is about 55 nm. For the top plate 110, it undergoes a simple hydrophobic layer coating process for the top hydrophobic layer 112.

In one embodiment, in addition to glass, possible substrates may include rigid or flexible ceramic, semiconductor, metal, polymer, composite materials, printed circuit board (PCB), flexible printed circuit board (FPC), etc.

In another embodiment, elementary essential oils including orange, rosewood and clove, respectively representing the top, middle and base notes of the fragrance are loaded onto its own reservoir with appropriate volume (e.g., 3 μL).

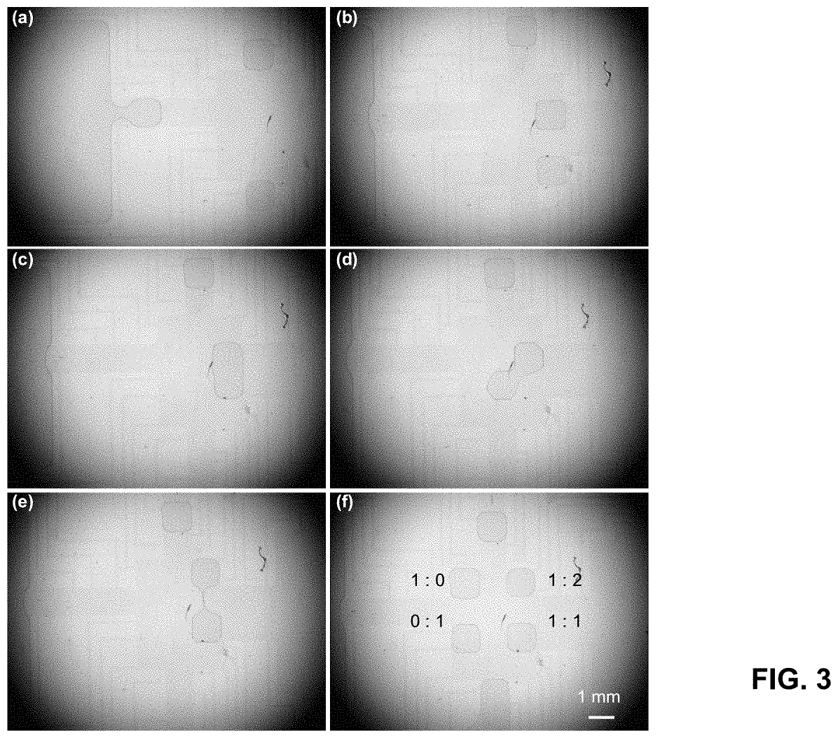

With 1 mm×1 mm driving electrodes 1212, 40 nL essential oil droplets can be individually generated in the 40 μm gap between the parallel plates (110, 120) by applying sufficient voltages. As shown in FIG. 3a, a top note orange essential oil droplet (volume 40 nL) is generated from the left reservoir. It is transported to the right above a base note clove essential oil droplet (volume 40 nL) generated from the lower reservoir as shown in FIG. 3b. The top reservoir accommodates the rosewood essential oil. The orange and clove essential oil droplets are merged and mixed along a loop driving path to enhance the mixing as shown in FIGS. 3c and 3d. The mixture droplet is split into two 40 nL droplets whose orange to clove essential oil ratio is 1:1. Different orange to clove ratios are readily achievable by mixing droplets at desirable proportions. For example, as shown in FIGS. 3e and 3f, the ratios of 1:0, 1:2, 0:1 and 1:1 are obtained after orange and clove essential oil droplets generation, mixing and splitting. Thus, an arbitrary fragrance composed of encoded elementary essential oils can be digitally programmable on the EMF platform 100 in the present invention.

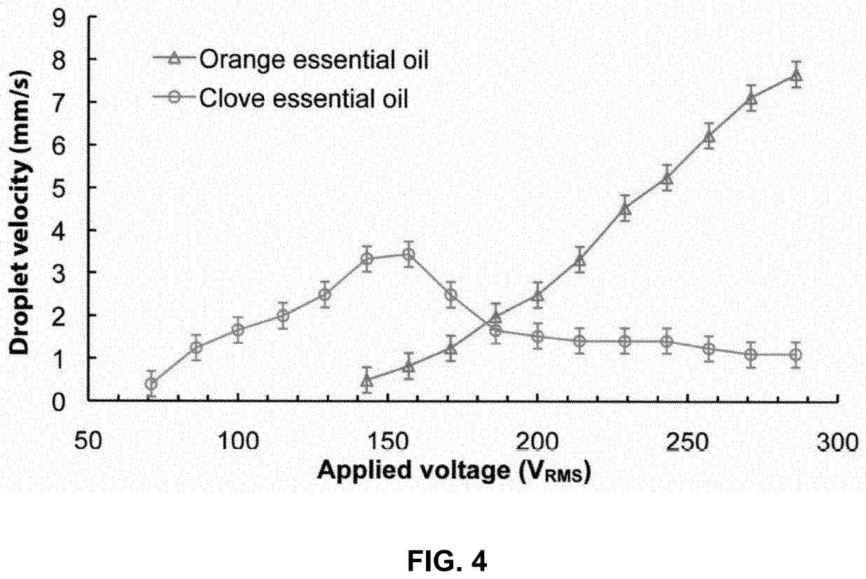

The driving ability of the 40 nL essential oil droplets 130 on the EMF platform 100 is demonstrated with droplet velocity at different voltages in FIG. 4. The major component of the orange and clove essential oils are limonene (relative permittivity 2.3, viscosity 0.9 cP) and eugenol (relative permittivity 3.4, viscosity 4.5 cP), respectively.

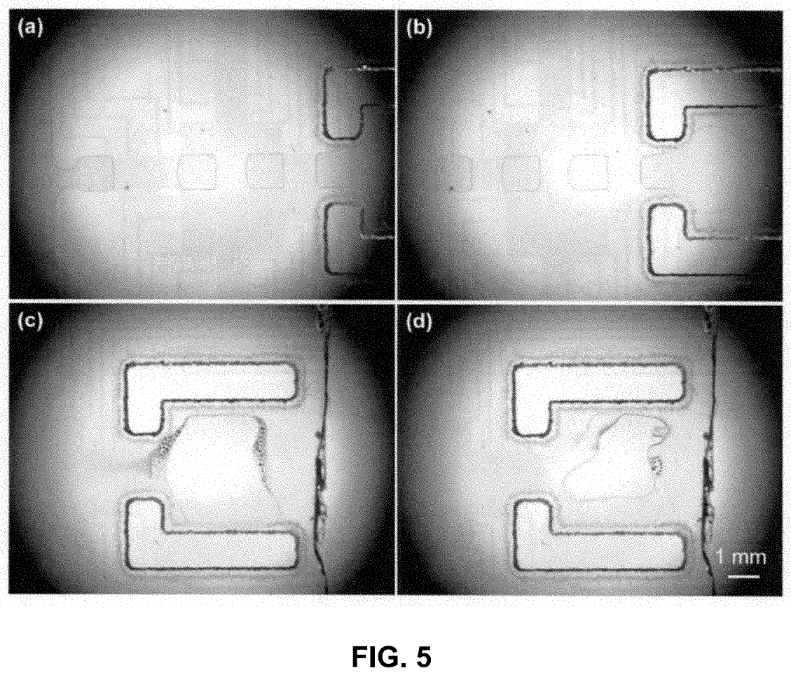

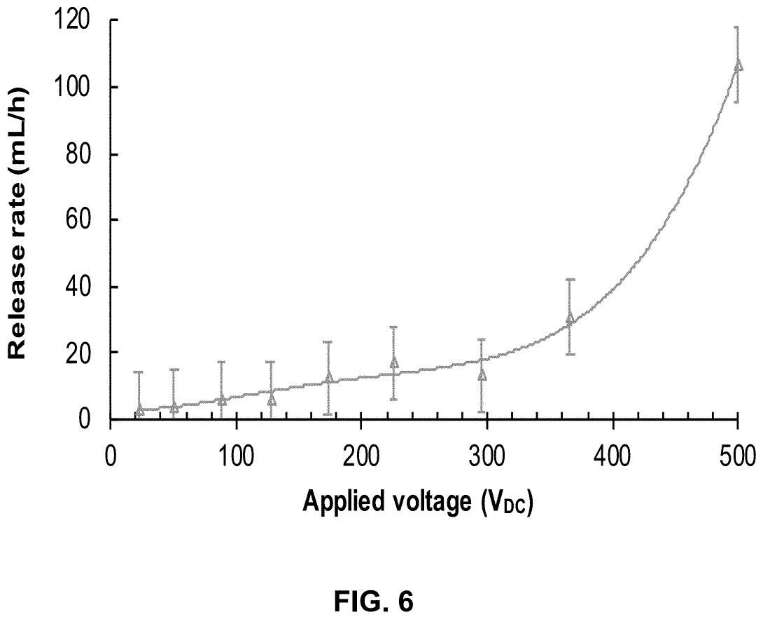

FIGS. 5a and 5b demonstrate the preparation of a larger essential droplet by mixing eight 40 nL droplets. The droplets are merged on the microheater electrodes 1213 by dielectrophoresis when applying an appropriate equal electric potential on the two terminals of the microheater electrodes 1213. Subsequently, a proper electric potential is applied between the two terminals and generated heat on the microheater. The evaporation of the merged droplet (volume 320 nL) is adjustable based on the supplied power to the microheaters. The fragrance releases via the two corresponding L-shaped openings through the top glass plate. The fragrance releasing is highly programmable at an adjustable release rate, for example 110 mL/h shown in FIG. 6. The resistances of the microheaters are tunable based on the demand for the release rate.

In another aspect, a programmable fragrance generating apparatus 200 may include a control center 210, a plurality of fragrance receiving holes 220, a receiving space 230 for at least one EMF platform 100, and a programmable fragrance releasing outlet 240, wherein the EMF platform is electrically connected with the control center 210 and has a plurality of reservoirs 1211 to receive different liquid fragrances from the fragrance receiving holes 220; each liquid fragrance is controlled by the control center to transport from each reservoir to one or more driving electrodes 1212 on the EMF platform to mix with one or more other liquid fragrances, which can be further transported to one or more heating electrodes 1213, atomizers, or nebulizers for fragrance releasing by evaporation at a predetermined temperature.

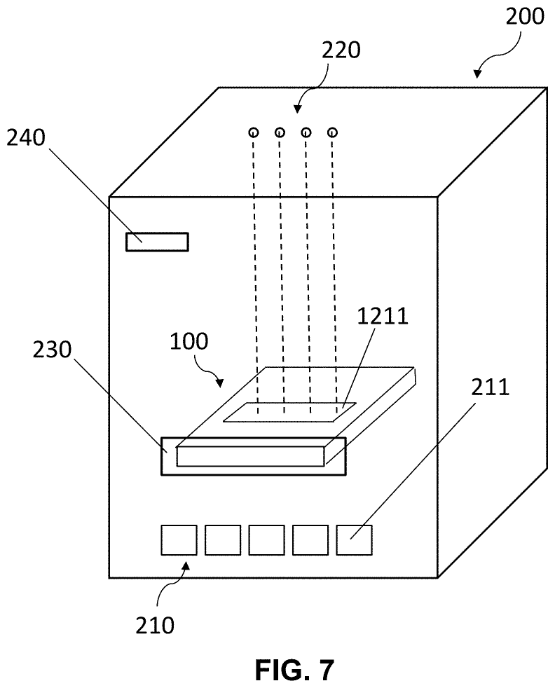

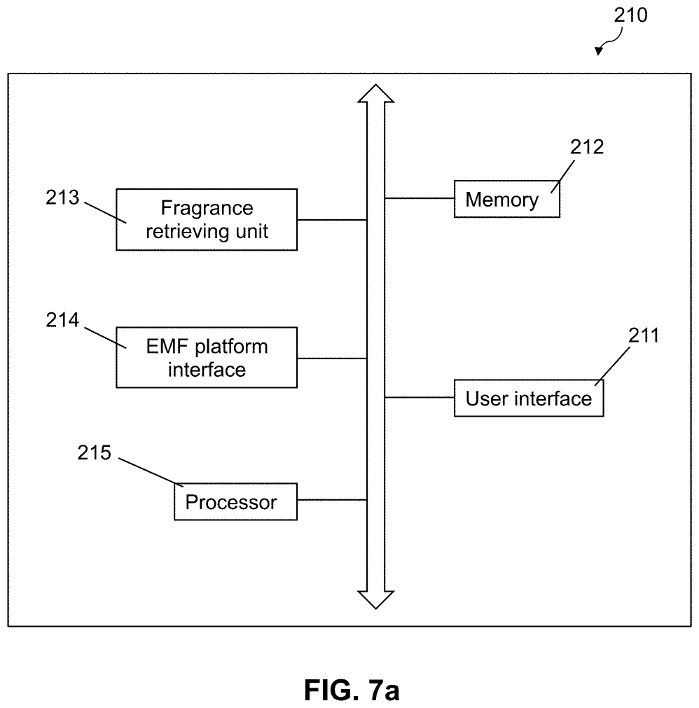

In an exemplary embodiment, as shown in FIG. 7a, the control center may include the user interface 211, a memory 212, a fragrance retrieving unit 213, an EMF platform interface 214 and a processor 215. In one embodiment, the fragrance retrieving unit 213 may be operatively communicate with the user interface 211, and when the user operates the user interface 211 to program at least one of the fragrances receiving holes 220, the fragrance retrieving unit 213 is configured to introduce and transport the fragrance(s) selected by the user to the reservoir 1211 on the EMF platform 100.

The EMF platform interface 214 and the processor 215 are configured to communicatively and electrically connect with the EMF platform 100, and the processor 215 is configured to execute an instruction received from the user interface 211 to generate one specific fragrance or a specific ratio of the mixture of two or more fragrance. More specifically, the processor 215 is configured to drive one or more fragrances to the driving electrodes 1212 to digitally program the fragrance(s) by either splitting, mixing, or combining the fragrances thereat according to the instruction, and then transport the programmed fragrance to the microheater electrodes 1213 to generate the fragrance as instructed by the user.

In one embodiment, the liquid fragrance is driven by electrical force and the volume thereof can be determined by an arrangement of the electrodes on the EMF platform 100 and controlled by the control center 210. It is noted that the electrical force can be dielectrophoresis force. The driving force can also be electrowetting if the liquid fragrance does not contain oil.

In another embodiment, a cleaning liquid can be disposed at one of the fragrance receiving holes and can be controlled by the control center to be electrically transported into the EMF platform 100 to wash the electrodes. In a further embodiment, the EMF cartridge can be replaced after being washed by predetermined times.

In still a further embodiment, as discussed above, the EMF platform 100 may have two parallel plates; the bottom plate 120 may include patterned electrodes 121 covered by dielectric and hydrophobic layers, and the top plate 110 may have an unpatterned electrode 111 covered by a hydrophobic coating. It is important to note that the EMF droplet manipulation platform 100 in the present invention can also be used in aromatherapy or other medical fields such as breathing treatments for respiratory disease or disorders.

Having described the invention by the description and illustrations above, it should be understood that these are exemplary of the invention and are not to be considered as limiting. Accordingly, the invention is not to be considered as limited by the foregoing description, but includes any equivalent.

What is claimed is:

1. A programmable fragrance generating apparatus comprising:
a receiving space to receive at least one electro-microfluidic (EMF) platform that includes a top plate and a bottom plate that are separated to provide the fragrance an operation gap therebetween;
a plurality of fragrance receiving holes;
a control center communicatively and electrically connected to the EMF platform, said control center having a user interface to receive an instruction to generate a programmed fragrance; and
a fragrance releasing outlet,
wherein the EMF platform receives at least one fragrance from the fragrance receiving holes, and the EMF platform includes electrodes associated with the top plate and the bottom plate to transport said fragrance; and the control center is configured to program the fragrance on the EMF platform to digitally generate the programmed fragrance according to the instruction.

2. The programmable fragrance generating apparatus of claim 1, wherein the electrodes in the EMF platform include reservoir electrodes, driving electrodes and microheater electrodes.

3. The programmable fragrance generating apparatus of claim 2, wherein the reservoir electrodes are configured to receive the fragrance(s) from the fragrance receiving holes.

4. The programmable fragrance generating apparatus of claim 2, wherein the control center is configured to program the fragrance(s) at the driving electrodes by either splitting, mixing, or combining the fragrances according to the instruction.

5. The programmable fragrance generating apparatus of claim 4, wherein the programmed fragrance(s) is transported to the microheater electrodes where the electrodes are heated to evaporate and release the fragrance(s) through the fragrance releasing outlet.

6. The programmable fragrance generating apparatus of claim 1, wherein the top plate and bottom plate are made by glass.

7. The programmable fragrance generating apparatus of claim 1, wherein the programmed fragrance can be released through atomizers or nebulizers integrated on the EMF platform.
